(19) United States
(12) Patent Application Publication
(10) Pub. No.: US 2012/0289862 A1
(43) Pub. Date: Nov. 15, 2012

(54) PERFORATED ACCESS SHEATH FOR PERCUTANEOUS MEDICAL PROCEDURES

(76) Inventor: Ali Shariati, Lakeland, FL (US)

(21) Appl. No.: 13/106,164

(22) Filed: May 12, 2011

(51) Int. Cl. A61B 10/02; A61M 5/00

(52) U.S. Cl. 600/567; 604/272

(57) ABSTRACT

A method and a device are disclosed for the treatment of pneumothorax (air leak) or other similar medical pathologic conditions necessitating percutaneous (through skin) access by a channeling or conduit device, such as a syringe or tube. In various embodiments, a flexible sheath with peripheral holes, an entry orifice, and an end hole is used to enclose and channel a guide needle or other channeling device to evacuate air or fluid from around organs or within body cavities. The flexible sheath may further be used for providing access by relatively larger tubes into body cavities. Still further, the flexible sheath may be used for infusing blood or other coagulant to seal trauma sites within body cavities, or to deliver medicine or other material to a target site within the body.

### TECHNICAL FIELD

This application relates generally to medical biopsy procedures. More specifically, this application relates to a method and apparatus for treating pneumothorax.

### BRIEF DESCRIPTION OF THE DRAWINGS

The drawings, when considered in connection with the following description, are presented for the purpose of facilitating an understanding of the subject matter sought to be protected.

FIG. 1 shows an example patient undergoing a medical biopsy procedure;

FIG. 2 shows an example coaxial system configured for use in medical procedure;

FIGS. 3A and 3B show an example guide needle and perforated sheath;

FIG. 4 shows an example biopsy procedure of a mass in lung tissue; and

FIGS. 5A-5D show example lung biopsy procedures and treatments of resulting pneumothorax.

### DETAILED DESCRIPTION

While the present disclosure is described with reference to several illustrative embodiments described herein, it should be clear that the present disclosure should not be limited to such embodiments. Therefore, the description of the embodiments provided herein is illustrative of the present disclosure and should not limit the scope of the disclosure as claimed. In addition, while the following description references lung biopsy, it will be appreciated that the disclosure may be used with other types of medical procedure such as biopsy of other organs, injection and/or delivery of various drugs and fluids in various body cavities like the abdominal cavity, and the like.

Briefly described, a method and a device are disclosed for the treatment of pneumothorax (air leak) or other similar medical pathologic conditions necessitating percutaneous (through skin) access by a channeling or conduit device, such as a syringe or tube. In various embodiments, a flexible sheath with peripheral holes, an entry orifice, and an end hole is used to enclose and channel a guide needle or other channeling device to evacuate air or fluid from around organs or within body cavities. The flexible sheath may further be used for providing access by relatively larger tubes into body cavities. Still further, the flexible sheath may be used for infusing blood or other coagulant to seal trauma sites within body cavities, or to deliver medicine or other material to a target site within the body.

Many medical procedures, such as biopsies, may have side effects, which may need to be treated as a separate medical problem. For example, small pneumothoraces (air leaks) that may occur after lung biopsies are treated by evacuating the air from the pleural space (space surrounding the lungs between the lung and chest wall) and repairing the perforations, which allowed air to infiltrate the pleural space. Often, these procedures may be uncomfortable and painful for patients and a source of stress to both patient and physician. Generally, such pneumothoraces may be treated by re-sticking the patient with another needle to access the pleural space if the air leak (pneumothorax) is large enough. If the pneumothorax is not sufficiently large for this procedure, then they are carefully monitored until healed. The pneumothorax is sometimes evacuated by the guide needle itself on the way out, but this has the added danger of creating another hole in the lung as the lung re-expands to approximate the chest wall again.

Normally, the pressure in the lungs is greater than the pressure in the pleural space surrounding the lungs. However, if air enters the pleural space, the pressure in the pleura then becomes greater than the pressure in the lungs, causing the lung to collapse partially or completely. Pneumothorax may be either spontaneous or due to trauma. If a pneumothorax occurs suddenly or for no known reason, it is called a spontaneous pneumothorax. In addition, people with lung disorders, such as emphysema, cystic fibrosis, and tuberculosis, are at higher risk for spontaneous pneumothorax. Traumatic pneumothorax is the result of accident or injury due to medical procedures performed to the chest cavity, such as lung biopsy, thoracentesis, or mechanical ventilation. Tension pneumothorax is a serious and potentially life-threatening condition that may be caused by traumatic injury, chronic lung disease, or as a complication of a medical procedure. In this type of pneumothorax, air enters the chest cavity, but cannot escape. This greatly increased pressure in the pleural space causes the lung to collapse completely, compresses the heart, and pushes the heart and associated blood vessels toward the unaffected side. Therefore, a new procedure and/or device is needed to avoid or reduce such additional trauma and health risks.

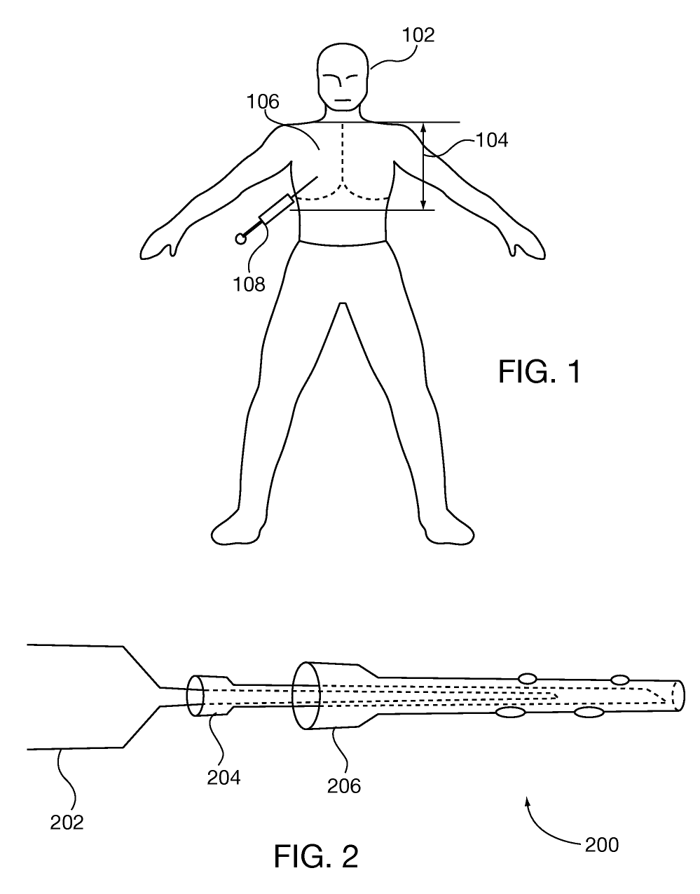

FIG. 1 shows an example patient undergoing a medical biopsy procedure. Patient 102 undergoes a medical procedure in the lung area 106 located in thorax 104 using a medical device 108. For example in a coaxial percutaneous lung biopsy, a guide needle may be used. A typical medical device 108 used for such procedure is a Temno coaxial system with sampling needle. Those skilled in the art will appreciate that similar procedures may be employed for biopsy of other parts of the anatomy for various purposes. For example, similar medical procedures may be used to percutaneously access bladder, uterus, abdominal cavity, other body cavities, and the like for various medical purposes, such as drug delivery, diagnostic, repair, and the like.

FIG. 2 shows an example coaxial system configured for use in medical procedure. In various embodiments coaxial system 200 includes perforated flexible sheath 206 is configured to allow a conduit such as guide needle 204 pass through perforated flexible sheath 206, and medical apparatus, such as a needle of syringe 202 pass through guide needle 204.

In various embodiments, perforated flexible sheath 206 is constructed from various biologically inert materials such as some plastics, carbon fibers, various gas permeable and non-gas permeable polymers, vinyl, and the like. In various embodiments, coaxial system 200 may include fewer or more coaxial components. For example, perforated flexible sheath 206 may include two layers, one embedded within the other, to reinforce the sheath, adjust the internal diameter of the sheath, or for other purposes. Similarly, guide needle 204 may include more than one concentric layer.

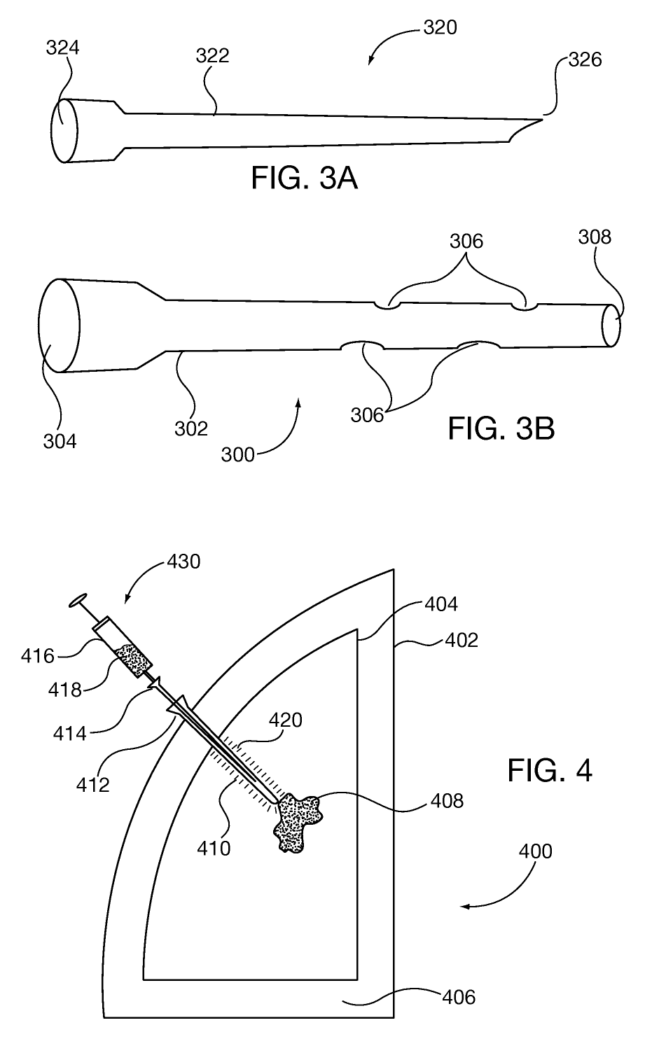

FIGS. 3A and 3B show an example guide needle and perforated sheath. FIG. 3A shows a guide needle 320 including shaft 322, entry orifice 324 configured to receive a medical device such as a syringe needle, and tip 326. In various embodiments, guide needle 320 is configured to fit through perforated flexible sheath 300 (shown in FIG. 3B). Guide needle 320 is generally configured to receive and guide a working needle, such as a syringe needle, to deliver and/or evacuate air or fluids to/from a target body site, respectively.

In various embodiments, shaft 322 and entry orifice 324 have a circular cross sections, while in other embodiments, shaft 322 and entry orifice 324 have other cross sectional shapes, such as oval, rectangular, U-shaped, or other shapes for various applications and target body sites. For example, a flat cross section may be more suitable and less intrusive than a circular cross section for accessing some organs or cavities within the body. In various embodiments, cross sectional shape of entry orifice 324 is different from the cross sectional shape of shaft 322. For example, entry orifice 324 cross section may be circular, while shaft 322 cross section may be oval.

FIG. 3B shows perforated flexible sheath 300 including perforated shaft 302 having peripheral holes 306, entry orifice 304, and tip hole 308, all connected to each other via a hollow space within the perforated shaft. In various embodiments, entry orifice 304 is configured to receive guide needle 320 to pass through tip hole 308 and access a target body cavity. In various embodiments, guide needle 320 and peripheral holes 306 are configured to allow guide needle 320 to substantially block peripheral holes 306 and prevent fluid flow through holes 306.

In various embodiments, in a manner similar to guide needle 320, shaft 302 and entry orifice 304 have a circular cross sections, while in other embodiments, shaft 302 and entry orifice 304 have other cross sectional shapes, such as oval, rectangular, U-shaped, or other shapes for various applications and target body sites. For example, a flat cross section may be more suitable and less intrusive than a circular cross section for accessing some organs or cavities within the body. In various embodiments, cross sectional shape of entry orifice 304 is different from the cross sectional shape of shaft 302. For example, entry orifice 304 cross section may be circular, while shaft 302 cross section may be oval.

Perforated flexible sheath 300 generally fits over guide needle 320. Perforated flexible sheath 300 may be used for several purposes including receiving and guiding a guide needle or other channeling device to evacuate air or fluid from target body sites, such as around organs or within body cavities, providing access using relatively larger tubes into body cavities for fluid drainage purposes, and infusing blood or other coagulant to seal trauma sites within body cavities, or to deliver medicine or other material to a target site within the body.

After a lung biopsy has been performed, if there is a pneumothorax, the flexible sheath can be used on the way out of the lung parenchyma (tissue characteristic of an organ, as distinguished from associated connective or supporting tissues), to drain the air in the pleural space. The sheath may be left in place and secured to the skin as a temporary channel for access to the pleural space. This way, if pneumothorax recurs within a short period of time, such as in one or two hours, access to the pleural space already exists through the sheath. The channel created by the sheath may be dilated over a wire and a larger chest tube may be placed for drainage of air and fluids. If there is no pneumothorax after two hours, the sheath is simply pulled out. The sheath may also be used during and at the termination of the procedure to administer blood, saline, or other substance, which may aid in plugging the pleural hole and additionally the injured alveoli and bronchioles. The injection of blood into the sheath through the needle may be made as the needle is being advanced into the lung or as it is being removed from the lung. The guide needle may be pulled back slightly to uncover peripheral holes 306 near the tip hole 308 of the sheath, and then the blood or other coagulant may be injected. Presence of multiple peripheral holes, as opposed to one end hole (tip hole 308), may provide for better area coverage and better seal within the target body site.

FIG. 4 shows an example biopsy procedure of a mass in lung tissue. Thorax area 400 includes chest wall 402, lung 404, pleural space 406, and tissue mass 408. Coaxial system 430 including perforated flexible sheet 412, guide needle 414, syringe 416 having contents 418, may be used to sample tissue mass 408 for biopsy, and infuse target body site 410 with contents 418.

During the biopsy procedure, upon advancing and/or withdrawing the coaxial system from target body side 410, blood or other substance may be injected into the lung through peripheral holes 306 (shown in FIG. 3B) to infiltrate the damaged alveoli. To perform this operation, the guide needle may have to be withdrawn slightly to uncover the peripheral holes and tip hole of the sheath.

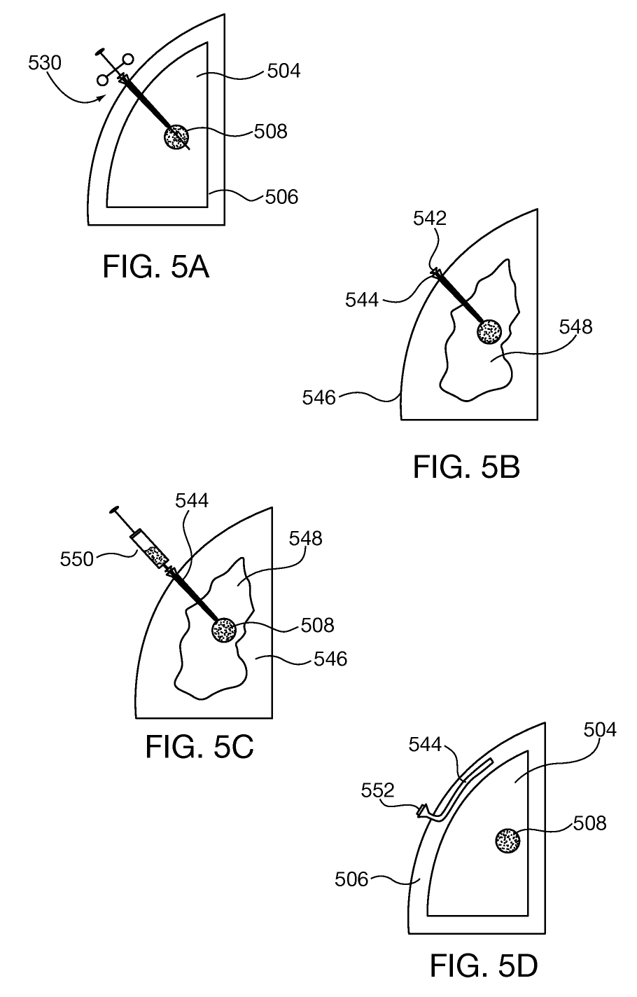

FIGS. 5A-5D show example lung biopsy procedures and treatments of resulting pneumothorax. FIG. 5A shows an initial phase of a biopsy in which coaxial system 530 may be used to retrieve a sample from a tissue mass 508 within lung 504 surrounded by pleural space 506.

FIG. 5B shows a next phase during in which tissue sampling needle is withdrawn after collecting the biopsy sample from lung 548, leaving behind guide needle 542 and perforated flexible sheath 544 within the target body site. Lung 548 has partially or completely collapsed due to traumatic pneumothorax induced by coaxial system 530 and pleural space 546 has grown in size.

FIG. 5C shows a next phase in the procedure for the treatment of pneumothorax. A syringe 550 is inserted into perforated flexible sheath 544 already inserted through pleural space 506 and after removal of guide needle 542. With the sheath pulled back from within lung 548 into pleural space 506, pneumothorax may be evacuated via syringe 550.

FIG. 5D shows a next phase in the procedure for the treatment of pneumothorax. In this phase, after the pneumothorax has been evacuated, lung 548 can re-expand. Perforated flexible sheath 544 may be left in pleural space to allow access if a larger chest tube is needed for drainage or for other purposes, such as endoscopic observations. A plug 552 may be inserted to block perforated flexible sheath 544 to prevent entry of air, particles, or germs.

While the present disclosure has been described in connection with what is considered the most practical and preferred embodiment, it is understood that this disclosure is not limited to the disclosed embodiments, but is intended to cover various arrangements included within the spirit and scope of the broadest interpretation so as to encompass all such modifications and equivalent arrangements.

I claim:

1. A medical sheath comprising:
an entry orifice;
a flexible shaft coupled with the entry orifice;
a peripheral hole in the flexible shaft; and
a tip hole at the end of the flexible shaft.

2. The medical sheath of claim 1, further comprising a hollow space connecting the entry orifice, the peripheral hole, and the tip hole.

3. The medical sheath of claim 1, wherein the entry orifice is configured to receive a guide needle.

4. The medical sheath of claim 3, wherein the peripheral hole is configured to be blocked by the guide needle.

5. The medical sheath of claim 3, wherein the peripheral hole is configured to allow fluid flow when the guide needle is pulled away from the peripheral hole.

6. The medical sheath of claim 1, wherein the flexible shaft is constructed from one of a polymer, vinyl, and carbon fiber.

7. The medical sheath of claim 1, wherein a cross section of the flexible shaft is substantially one of circular, rectangular, and oval.

8. The medical sheath of claim 3, wherein a cross section of the entry orifice is substantially the same as a cross section of the flexible shaft.

9. A method of performing biopsy, the method comprising:
using a coaxial system including a perforated flexible sheath to sample a tissue mass percutaneously.

10. The method of claim 9, further comprising retracting a sampling needle from the perforated flexible sheath to unblock a peripheral hole in the perforated flexible sheath.

11. The method of claim 9, wherein the biopsy comprises taking a sample from a lung.

12. The method of claim 11, further comprising evacuating a pleural space surrounding the lung via a peripheral hole in the perforated flexible sheath.

13. The method of claim 9, further comprising injecting a coagulating substance through a peripheral hole in the perforated flexible sheath.

14. The method of claim 9, further comprising leaving the perforated flexible sheath within a pleural space after completion of the biopsy.

15. A coaxial system comprising:
a perforated flexible sheath;
a guide needle configured to be inserted into the flexible perforated sheath; and
a sampling needle configured to be inserted into the guide needle.

16. The coaxial system of claim 15, wherein the perforated flexible sheath comprises an entry orifice, a flexible shaft, a peripheral hole, and a tip hole.

17. The coaxial system of claim 16, wherein the perforated flexible sheath further comprises a hollow space.

18. The coaxial system of claim 16, wherein the perforated flexible sheath is configured to be used to inject a substance into a body cavity via a peripheral hole in the perforated flexible sheath.

19. The coaxial system of claim 16, wherein the perforated flexible sheath is configured to be used to evacuate a pleural space resulting from a pneumothorax.

20. The coaxial system of claim 19, wherein the perforated flexible sheath is configured to be left in the pleural space.
